(19) United States
(12) Patent Application Publication
(10) Pub. No.: US 2007/0009961 A1
(43) Pub. Date: Jan. 11, 2007

(54) Identification of targeting component of herbal medicines from simplified HPLC spectrum using after flowing through immobilized receptor (AFTIR) method

(75) Inventors: Hueih-Min Chen, Shien Tein City (TW); Guo-Chung Dong, Changhua City (TW)

Correspondence Address: COHEN, PONTANI, LIEBERMAN & PAVANE, 551 FIFTH AVENUE, SUITE 1210, NEW YORK, NY, 10176, US

(73) Assignee: Academia Sinica

(21) Appl. No.: 11/176,139

(22) Filed: Jul. 7, 2005

(51) Int. Cl. G01N 33/53; G01N 33/567; A61K 36/18

(52) U.S. Cl. 435/7.1; 435/7.2; 424/725; 977/900

(57) ABSTRACT

A novel method of screening herbal composition for a pharmaceutically active component of interest (“target component”) comprises: immobilizing a plurality of receptors of interest on a chip; flowing the herbal composition through the chip to allow binding of the target component with the receptors; disassociating the bound target component from the immobilized receptors on the chip; profiling the disassociated target component by HPLC; and identifying the target component based on the HPLC profile of the disassociated target component.

### BACKGROUND OF THE INVENTION

1. Field of the Invention

The present invention relates to a novel method of screening herbal composition for a pharmaceutically active component of interest (“target component”), in particular to a method of identifying the target component contained in the herbal composition through the use of a receptor of interest.

2. Description of the Related Art

Herbal remedy, the use of plants to treat ill health, is the most ancient form of medication. It is a major practice in the developing world whilst rapidly gaining popularity in the industrialized nations, perhaps because of its reputedly low side effect profile. Its credibility is hotly contested with detractors highlighting its principal foundation upon empirical observation as opposed to rigorous, randomized, double-blind, placebo-controlled trials. Despite this, there can be no doubt that it has given much to modern medical practice, with many of the pharmaceuticals currently available to western physicians having a long history as herbal remedies. Similarly, there is a wide-ranging consensus that it still has so much to offer; herbal remedies being regarded as untapped pharmaceutical repositories.

Herbal medicines are complex mixtures containing up to thousands of compounds, only a minority of which are likely to have bioactivity. To discover herbal medicine's full potential, a high throughput drug screening initiative is required to isolate active ingredients from the 20,000 available herbal products. However, current screening methods carry heavy flaws. They comprise steps: (1) solvent extraction; (2) high performance liquid chromatography (HPLC) or gas chromatography (GC) spectrum profiling; (3) major peak collection and identification by mass spectroscopy; (4) structural identification by nuclear magnetic resonance (NMR); (5) bioactivity assay. This methodology assumes that an active ingredient will be an outstanding peak in the mass spectrum, which may not be the case, and much inefficiency comes from the bioactivity not being assayed at an early time.

Many membrane receptors of human cells have been found since 1960. The study for these receptors found that they play vital roles in mediating intracellular signal transduction via the binding (either strong or weak) with ligand(s). One point is then raised: if the binding between the specific ligand and the receptor is blocked, the signal transduction in cell will be confined. This blocking action can be therefore considered as a strategy to treat diseases related to the obstruction of this pathway. For example, antibodies of IL-2 (anti-IL-2) and TNF-alpha (anti-TNF-alpha) have been applied to block the interaction of cytokines (ligand) and its receptor. These blockings caused by anti-IL-2 and anti-TNF-alpha provides the useful way to treat with inflammation and hypersensitivity, respectively. However, uses of antibodies for treatment with these diseases are still clinically controversial. Development of new suppressants is therefore highly expected.

The binding concept applied to screen the targeted ligand(s) from mixture (but not herbal medicines) was done before by using affinity column (AC) with a receptor-base static phase (see, e.g., U.S. Pat. No. 6,607,921 and International patent application WO 97/43301.) However, the sample source suitable for this method is limited. In addition, the efficacy of this method is low. The static phase in the column generally contains spherical type of particles for immobilizing the receptors. The binding orientation of the receptors in column is not much homological but along multi-way directions. Therefore, the efficiency of immobilized receptors to bind the target ligands in the column is low. In addition, the target compound(s) is chemically identified by mass spectrum, which is connected immediately after the affinity column. In order to identify the target compound(s) based on the known database, the test sample, which is required by AC, should to be simple. Therefore, AC method is only suitable for a simple mixture, rather than any complex mixture such as an herbal medicine.

Accordingly, there is a need to develop a simple, efficient method of identifying a target component from an herbal composition.

### SUMMARY OF THE INVENTION

The present invention provides a method of identifying a target component of an herbal composition comprising:

immobilizing a plurality of receptors of interest on a chip;

flowing the herbal composition through the chip to allow binding of the target component with the receptors;

disassociating the bound target component from the immobilized receptors on the chip;

profiling the disassociated target component by HPLC; and

identifying the target component based on the HPLC profile of the disassociated target component.

Preferably, the chip is a glass-base biochip with micro-flowing pathways. The receptors are immobilized on the chip with homology of orientation. The chip may be a micro-array chip on which different types of receptor are immobilized. The chip may be reused for binding the target component of the herbal composition after the bound target component is disassociated with the immobilized receptors on the chip. The chip may be Sensor Chip CM5, CM4, CM3, C1, L1, SA, NTA, HPA, or Au, preferably, Sensor Chip CM5.

The receptors may be covalently immobilized on the chip. For example, the chip may be coated with a dextran having carboxyl group. Each of the receptors may contain a N-terminal amino group. Thus, the receptors are immobilized on the chip through the bond of the carboxyl group and the N-terminal amino group.

Preferably, the molar ratio of binding between the target component and the receptor is at least 0.9:1. The amount of the receptors may be as small as about 1 μg. The amount of the tested herbal crude extract may be as small as about 1.5 mg.

The method in accordance with the present invention may further comprise a step of identifying the target component by mass spectrometry, nuclear magnetic resonance, or combination thereof, subsequent to the identifying step by HPLC.

The herbal composition may also be profiled by HPLC before being flowed through the immobilized chip (“crude HPLC profile”). Later, the crude HPLC profile may be compared with the HPLC profile of the disassociated target component (“simplified HPLC profile”) to determine the peak corresponding to the target component in the crude HPLC profile of the herbal composition.

The receptors of interest may be selected from the group consisting of proteins, including recombinant proteins, glycoporteins, glycosaminoglycans, proteoglycans, integrins, enzymes, lectins, cell-adhesion molecules, toxins, bacterial pili, transport proteins, receptors involved in signal transduction, or hormorne-binding, hormones, antibodies, major histocompatability complexes, immunolgobulin superfamilies, cadherins, DNA or DNA fragments, RNA and RNA fragments, whole cells, cell fragments, tissues, bacteria, fungi, viruses, parasites, preons, and synthetic analogs, or derivatives thereof. For example, the receptors may be those that are involved in immunity, inflammation, and cancer, etc. Preferably, receptors may be fused recombinant receptors, e.g., CD28-mulg, soluble fusion protein consisting of the extracellular (1 34aa) domain of human CD28 fused to murine IgG2a Fc (233 aa).

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1 is schematic depiction of “After Flowing Though Immobilized Receptor (‘AFTIR’)” in accordance with one embodiment of the present invention.

FIG. 2 illustrates modes of activation in T-cell.

FIG. 3 shows the results of immobilization of CD28 receptor on the chip.

FIG. 4 shows the binding results of crude extract of Echinacea (CEE) on the chip immobilized with CD28.

FIG. 5 shows the effects of CEE in blocking the binding between CD80 and the chip immobilized with CD28.

FIG. 6 illustrates HPLC profiles of CEE before and after AFTIR.

FIG. 7 shows compound isolation with HPLC profiling.

FIG. 8 illustrates Jurkat cell activation.

FIG. 9 shows in vitro immuno-suppressive action of CEE and compound No. 1 (“C1”).

FIG. 10 illustrates cytotoxicity of Crude Extract of Echinacea (CEE) and C1.

FIG. 11 shows the structure of C1 identified by mass spectrometry and NMR spectroscopy.

FIG. 12 shows a schematic comparison of the traditional screening method (TSM) and AFTIR in accordance with present invention.

FIG. 13 shows AFTIR by using a micro-array chip in accordance with one embodiment of the present invention.

FIG. 14 compares the designs of Affinity Column in the prior art and AFTIR in accordance with one embodiment of the present invention.

FIG. 15 shows HPLC profiles of bidens pilosa before and after AFTIR.

### DETAILED DESCRIPTION OF THE PRESENTLY PREFERRED EMBODIMENTS

As used herein, the abbreviations below have the following meanings.

AFTIR: after flowing through immobilized receptor; C1: compound 1; CEE: crude extract of Echinacea; IL-2: interleukin-2; imm-CD28: immobilized CD28; MTT: (-[4,5-Dimethylthiazol-2-yl]-2,5-diphenyltetrazolium bromide; PHA: phytohemaglutinin; PMA: phorbol 12-myristate 13-acetate; RU: response unit; SPR: surface plasma resonance; TSM: traditional screening method.

### EXAMPLE 1

Introduction

FTIR is a novel method of screening herbal extracts for potential pharmaceuticals (see the schematic depiction of FIG. 1). As a proof of principle it finds a candidate immunosuppressant, chlorogenic acid, in Echinacea purpurea. AFTIR identifies this constituent by its selective binding to chip immobilized CD28, a receptor instrumental to immune functioning. Chlorogenic acid is shown to be immunosuppressant in vitro. It downregulates CD28 dependent interleukin-2 (IL-2) expression in a T cell culture line.

As shown by FIG. 1, a physiologically important receptor species is immobilized on a CM5 chip. Herbal extract is then driven to flow over this chip. Any extract molecular species that can bind the receptor will become ensnared on the chip. Binding is detected by Surface Plasmon Resonance (SPR). Non-specific binders are washed across the chip. Any remaining binding species, being able to specifically bind this receptor In vitro, can be hypothesised to specifically bind this receptor In vivo and act pharmaceutically. They can be isolated and identified. This methodology can be iterated for differing immobilized receptor species and differing herbal extracts.

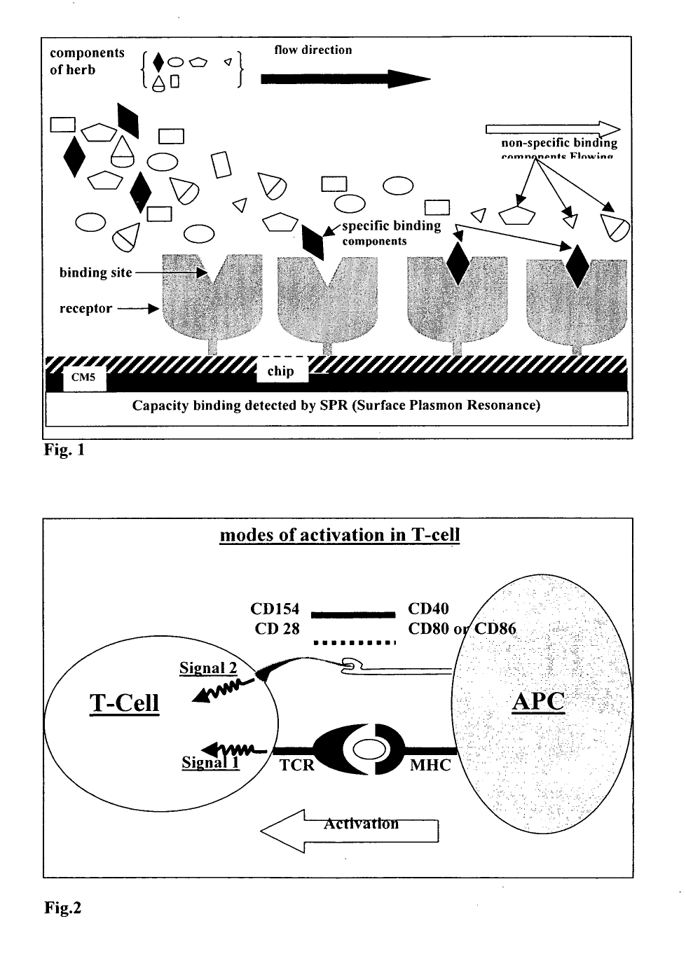

In the present study, we immobilize CD28 (imm-CD28) on a chip, flowing Echinacea purpurea extract across it. CD28 is a T-cell transmembrane receptor important to this cell class's differentiation to the “active” state, a state integral to the immune response and characterised by Interleukin-2 (IL-2) secretion (FIG. 2).

As shown by FIG. 2, T-cells differentiate to the “active” state, a state instrumental to the immune response, in retort to co-stimulation by two Antigen Presenting Cell (APC) signals 1) Major histocompatibility complex (MHC) (APC) strongly binds T-cell receptor (TCR) (T-cell); 2) [CD80 or CD86 (both APC) weakly binds CD28 (T-cell)] & [CD40 (APC) strongly binds CD154 (T-cell)].

Echinacea purpurea is a flower native to North America, and now popular as a western herbal medicine in Europe and America for its purported, yet heavily debated, boosting of the immune system.

Using AFTIR methodology, an Echinacea constituent compound was found to be able to selectively bind CD28. This compound can be envisaged to have an immuno-suppressive action in the body, perturbing physiological functioning of CD28. Presented In vitro studies support this hypothesis, with this compound downregulating CD28-dependent IL-2 expression in Jurkat cells, a T-cell culture line. Mass spectrometry and NMR spectroscopy identify this compound as chlorogenic acid. This compound is well studied but its immunosuppressive endowment is a novel finding. Echinacea purpurea also acts immunesuppressantly in this assay, constituent chlorogenic acid being the likely conferment. This finding is contrary to Echinacea's purported boosting of the immune system and therefore there is a need to reconsider its use given its popular use in the treatment of upper respiratory disorders, minor infections, herpes, candida, eczema and AIDS-related opportunistic infections.

Methods

Receptors and proteins. Human CD28-mulg and CD80-mulg fusion proteins were purchased from ID Labs Inc. CD28-mulg is a soluble fusion protein consisting of the extracellular (134 aa) domain of human CD28 fused to murine IgG2a Fc (233 aa). CD80-mulg is a soluble fusion protein consisting of the extracellular (173 aa) domain of human CD80 fused to murine IgG2a Fc (232 aa). Both were purified from tissue culture supernatant of CHO transfectants by protein A and size exclusion chromatography. Anti-human CD28 antibody and anti-human CD3 antibody were purchased from BioLegend. In the experiments, the quantity of anti-CD28 and CD80 used is only around 2% the quantity of CD28 immobilized.

Chemicals and Reagents. Sensor chip CM5, HEPES buffer solution HBS-EP (10 mM HEPES, 0.15 M NaCl, 3.4 mM EDTA, 0.05% surfactant P20, pH 7.4) and amine-coupling kit (N-hydroxysuccinimide (NHS), N-ethyl-N′-(3-diethylaminopropyl) carbodiimide (EDS) and ethanolamine hydrochloride) were obtained from Biocore AB (Uppsala, Sweden). Solvent used for chromatographic analysis was ChromAB grade of MeOH (Mallinckrodt, code no. 3041-68, suitable for liquid chromatography). Trifluoroacetic acid (TFA; 99% purity, Fluka) was used for eluent pH adjustment. Water used in this study was deionized and distilled. The chlorogenic acid reference standard was obtained from Fleton Reference Substance Co.,Ltd., Batch No. CH120041218, 98.4¢H. FK-506 and lonomycin were purchased from CalBiochem and both of them were dissolved by DMSO with concentration of 1 μg/ml. Phorbol 12-myristate 13-acetate (PMA) was purchased from Sigma. PMA was dissolved by DMSO and stored at −20° C. MTT (3-[4,5-Dimethylthiazol-2-yl]-2,5-diphenyltetrazolium bromide) was purchased from Sigma.

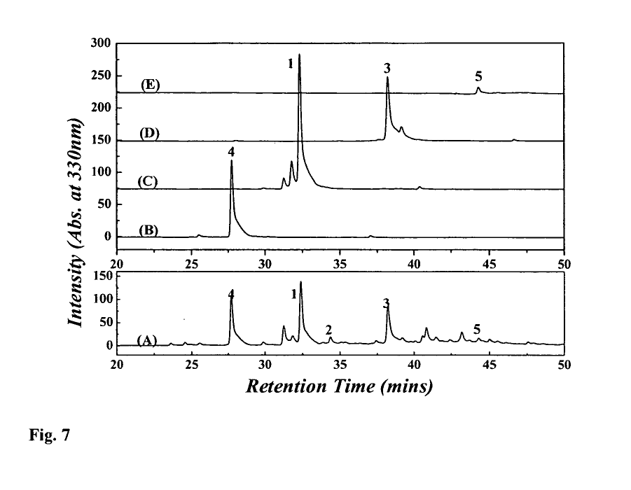

Preparations of CEE and target compounds. 50 g sample of Echinacea powder (Echinacea purpurea, Save-on-Albertson's™ Inc. USA) was extracted with 100% dd-H₂0 (1000 ml) by stirring at room temperature for 72 hours. The maceration extracts were collected and filtered through the filter-paper No. 1, code no. PW300-1125, pore size=10 μm, TOYO Inc. Extraction was repeated for three times. All filtrates were collected and concentrated to 500 ml under reduced pressure. 4000 ml acetone was added to the concentrate and the solution was stocked at −20° C. for 24 hours. Afterward, the solution was centrifuged at 8000 rpm for 60 min. The supernatant was collected and then completely dried under the reduced pressure. The final crude extract of Echinacea (CEE) was used for the AFTIR experiment. The target compounds such as C1 to C5 are collected due to the description shown in FIG. 7.

Surface plasmon resonance analysis. BIAcore 3000 (Biacore, Uppsala, Sweden) was employed for real-time biospecific interaction analysis. Binding analysis was performed at 25° C. with a flow rate of 10 μl/min. In general, proteins can be immobilized on the layer of carboxylated dextran in CM5 sensor chip (research grade; Biacore) by amine coupling. With each injection, samples were diluted in the running buffer HBS (10 mM HEPES, pH 7.4, 150 mM NaCl, 3.4 mM EDTA, 0.005% surfactant P20) to a final concentration of 15 to 30 μM. After each injection, flow-cell can be regenerated by flowing though the cell with the buffer containing 50 mM NaOH. Extracts were injected at different concentrations from 1.9 μM to 60 μM. The binding constant between immobilized receptor (imm-CD28) and ligand was determined using BIAevaluation 3.1 software (Biacore).

High performance liquid chromatography (HPLC) analysis. HPLC analysis was performed on the HP Technologies 1100 Series HPLC Modules with 250×4.6 mm Discovery BIO Wide Pore C18 HPLC Column (SUEPLCO, USA). The machine was connected to a diode array detector. Two organic solvents were applied as eluents: eluent A (99.95% H₂O/0.05% TFA (v/v)) and eluent B (99.95% MeOH/0.05% TFA (v/v)). Both eluents were filtered in vacuum and then degassed. Injection 20 Al of CEE into the column was chromatographed due to the program provided by manufacturer. The analysis of sample using solvent eluent was as follows: eluent A was injected first for 10 min and followed by a linear gradient from 0% to 100% of eluent B for over 60 min. Eluent B was continuously maintained for 10 min at flow rate 0.6 ml/min. The compound(s) of CEE eluted from the column was monitored at UV_{330 nm}. HPLC spectra were obtained and used for the comparison between before and after AFTIR experiments.

LC-MS Analysis. Atmospheric pressure ionization mass spectrometry analysis was performed on a Quattro LC benchtop triple quadrupole mass spectrometer (JASCO international, JAPAN). The machine was operated by electrospray ionization with a negative mode (ES⁻) interface. Mass spectrometric data are acquired in the full scan mode in the range of m/z between 150 and 600. Sensitivity of the mass spectrometer was optimized using the chlorogenic acid standard from Fleton Reference Substance Co., Ltd. Agilent 1100 HPLC system was used for the separation of samples and standards. The Luna C18(2) column and mobile phase gradient were identical to those used for RP-HPLC-UV analysis described above and 5 μL of C1 sample was injected per run by manual injector. The flow rate was fixed at 0.12 ml/min. The spectrometric analysis was done due to MassLynx 3.5 software (Micromass Ltd).

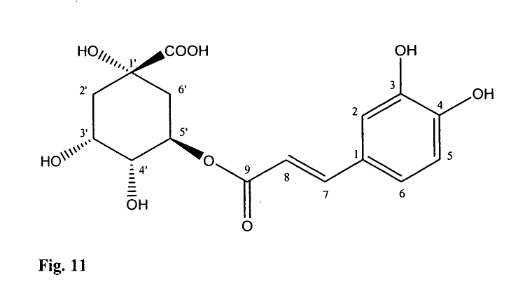

NMR Spectroscopy. C1 compound isolated and collected by chromatography (see the section of collection of C1 compound above) was dissolved in 0.8 mL of CD₃OD. ¹H and ¹³C NMR spectra were acquired at 25° C. by using Bruker QE 400 MHz NMR spectrometer. Chemical shift values were assigned relative to the frequencies of residual non-deuterated water and methanol externally referenced to tetramethylsilane. The chemical shifts of ¹H NMR (CD₃OD) spectrum of C1 compound were obtained as follows: δ: 2.03-2.13 (2H, m) and 2.16-2.28 (2H, m) [C2,6-H], 3.15 (1H, dd, J=3, 9 Hz, C4-H), 3.55 (1H, d, J=4 Hz, C3-H), 3.85 (1H, m, C5-H), 4.81 (2H, s, caff —OH), 6.18 (1H, d, J=16 Hz, caff C8-H), 6.74 (1H, d, J=8 Hz, caff C5-H), 6.89 (1H, dd, J=2, 8 Hz, caff C6-H), 7.00 (1H, d, J=2 Hz, caff C2-H), 7.49 (1H, d, J=16 Hz, caff C7-H). The chemical shifts of ¹³C NMR (CD₃OD) spectrum of C1 compound were as follows: δ□41.3 (C-2′), 30.0 (C-6′), 62.9 (C-5′), 59.5 (C-3′), 81.5 (C-4′), 72.3 (C-1′), 113.6 (caff C-2), 114.0 (caff C-8), 115.0 (caff C-5), 121.3 (caff C-6), 126.3 (caff C-1), 145.3 (caff C-7), 145.5 (caff C-3), 148.0 (caff C-4), 169.5 (caff C-9). Based on the results of mass spectrum and NMR assignments, the formula and structure of C1 compound was shown in FIG. 11.

Stimulation of T-cell. Jurkat leukemic T cells were maintained in a humidified atmosphere of 5% CO₂/95% air at 37° C. in RPMI-1640 medium (HyClone, Utah, USA) including penicillin, streptomycin and 10% heat-inactivated FBS. Two modes of activations were used for the present experiments: anti-CD3 plus anti-CD28 (CD28-dependent stimulation) and PMA plus ionomycin (CD28-independent stimulation). The detail pathways of these two modes are shown in FIG. 8. For experiment of CD28-dependant stimulation, flat-bottom 96-well plates were coated with 10 μg/ml of anti-CD3 for 24 h at 4° C. Wells anti-CD3 were washed twice with PBS. After washing, the unbounded anti-CD3 was removed. Jurkat T-cells (200 μl 2×10⁶ cells/ml) with soluble anti-CD28 (1 μg/ml) were then added into the wells. The cells were activated by anti-CD3 already existing in the wells. Upon the activation, the cell solution was stayed for 24 h. The supernatant (100 μl) of cell solution was then used for the measurement of IL-2 by enzyme-linked immuno-sorbent assay (ELISA). For experiment of CD28-indepentant stimulation, 200 μl Jurkat cell (5×10⁵ cell/ml) were incubated with PMA (50 nM)/lonomycin (1 μg/ml) for 24 h. 100 μl supernatant was then used for IL-2 measurement by ELISA. For experiment of CEE and C1 on T-cell, these materials were added into the culture plates including the cells and pre-incubated for 15 min before stimulation. Afterwards, the cells including CEE or C1 were stimulated via either CD28-dependant or CD28-independent stimulations. The culture supernatants in both modes were harvested after 24h incubation and then used for the measurements of IL-2. IL-2 measurement was done using the commercial available ELISA kit (Biosource; California, US). The details how to measure the concentration of IL-2 after T-cell is activated is shown below.

IL-2 ELISA assay. IL-2 concentration of T-cell was determined by following the instruction from manufacturer (Biosource; California, US). A 96-well flat-bottom plate was coated with anti-IL-2 mAb (100 μl at 4 μg/ml) in phosphate-buffered saline (PBS; pH 7.3) at room temperature overnight. The plate was then washed with PBS containing 0.05% Tween 20 (PBS-T) three times. Subsequently, the plate was incubated with a blocking solution containing 1% bovine serum albumin in PBS for more than 1 hr. After washing by PBS-T, 100 μl supernatant mixed with biotinylated anti-IL-2 detection antibodies (50 μl at 12.5 ng/ml) was added into each well and stay for 2 hr. After washing, 100 μl streptavidin horseradish peroxidase (1/2000 dilution of a 1.25 mg/ml solution) was added and the solution was incubated for 30 min at room temperature. After washing, 100 μl substrate solution containing 1/1 mixture of H₂O₂ and tetramethylbenzidine was added and the solution was incubated for 25min at room temperature. The solution reaction involving the stimulated sample was ceased by the addition of 100 μl stop solution (1M H₂SO₄). Concentration of IL-2 was then measured with microplate reader. The amount of IL-2 was indicated by the relative percentage (1200 pg/μl of IL-2 is defined as 100%). One-way ANOVA was used for both measurements of IL-2 production and cytotoxicity in T-cells induced by FK506, CEE and C1. The resultant data were treated by using superANOVA statistical software (Berkeley, Calif.) and can be accepted only if P value is smaller than 0.05.

MTT colorimetric assay. Cytotoxicity of T-cell during the stimulation induced by CEE or C1 compound was measured by MTT (3-[4,-Dimethylthiazol-2-yl]-2,5-diphenyl -tetrazolium bromide) colorimetric assay. 100 μl Jurkat cells (5×10⁵cells/ml) was incubated for 24 h with various concentrations of CEE and C1. Then, the cell solution was centrifuged at 200 g for 10 min and its supernatant was removed. 25 μl of MTT (5 mg/ml in H₂O) was added and the cell solution was again incubated at 37° C. for 4 hr. Afterwards, 200 μl of lysis buffer (DMSO) was added and the content of dissolved MTT crystals was measured with an ELISA reader (Dynatech, Chantilly, Va.). The survival rate (%) was determined as: OD_{560 nm} of test sample (CEE or C1)/OD_{560 nm} of control (PBS buffer only)×100%.

Results

Immobilization of CD28 Receptor on the Chip

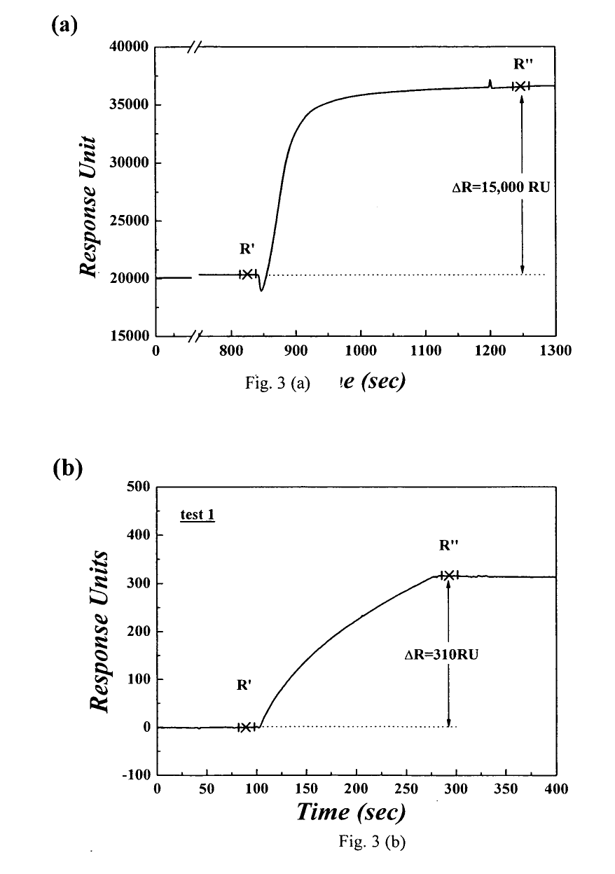

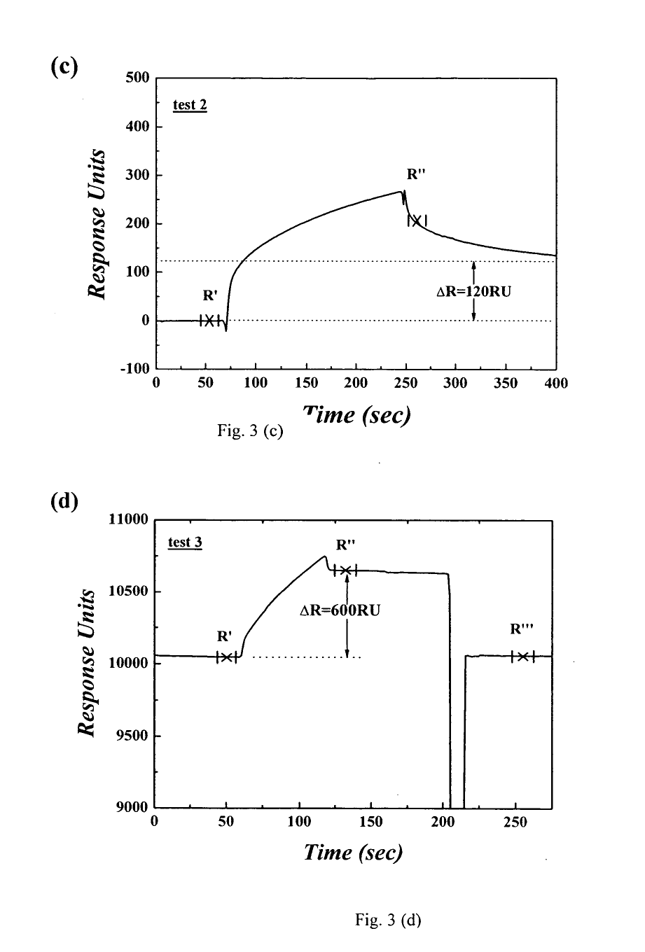

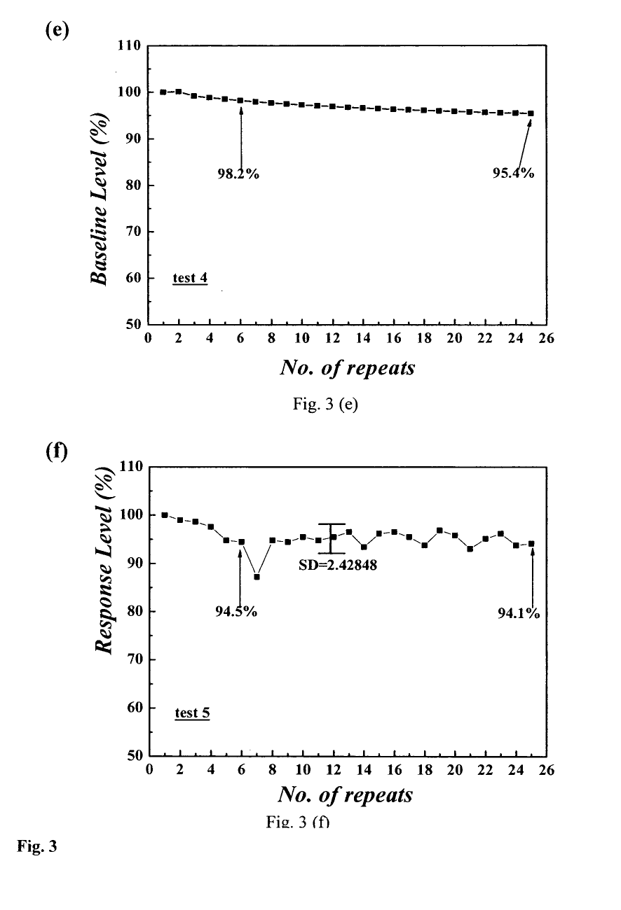

CD28-mulg, a soluble fusion protein consisting of the extracellular (134 aa) domain of human CD28 fused to murine IgG2a Fc (233 aa), was immobilized to dextran matrix on the sensor chip surface, via covalent coupling of primary amines (—NH2). In this report, the term CD28 will be used as shorthand for CD28-mulg. The strength of binding was assayed by the response unit (RU), using surface plasmon resonance (SPR; see Materials and Methods). FIG. 3a shows the corresponding sensorgram. The “binding capacity”, ΔR (ΔR=R″−R′; see FIG. 3a), of CD28 on the sensor chip was approximately 15.0 ng/mm² surface coverage (15,000 response units (RU); 1 RU=1 pg/mm²). CD28 is evidently well immobilized on the chip. When immobilized, CD28 retains its native binding activity. It can strongly bind its antibody (FIG. 3b. ΔR=˜310 RU with 10 μg/ml anti-CD28) and weakly bind CD80 (FIG. 3c. ΔR=˜120 RU with 10 μg/ml CD80).

AFTIR methodology relies not simply on selective molecular binding to the chip, but also on the ability to dissociate bound molecules for identification and reuse of the chip. Various washing solutions were tested, with 50 mM NaOH performing the best. FIG. 3d shows a typical sensorgram for anti-CD28 binding (ΔR=˜600 RU with 10 μg/ml anti-CD28) followed by NaOH washing solution injection (concentration=50 mM; flux=50 μl/min; time=10 sec). After washing, the anti-CD28 is largely disassociated, with the binding affinity almost equal to the pre-antibody application value (R′=˜R′″). One hundred cycles of this same anti-CD28 application and washing protocol were performed on the same chip. FIG. 3e plots R′″ as a percentage of R′, against iteration number, for 25 of these 100 iterations; R′″ is never less than 95.4% of R′ in this iteration range. FIG. 3f shows anti-CD28 binding capacity (ΔR) against iteration number. For 100 cycles mean ΔR=598 RU, with a relative standard deviation (RSD) of 3.4% (data not shown). Evidently, the chip is reusable.

Crude Extract of Echinacea (CEE) Binds CD28 Immobilised on the Chip

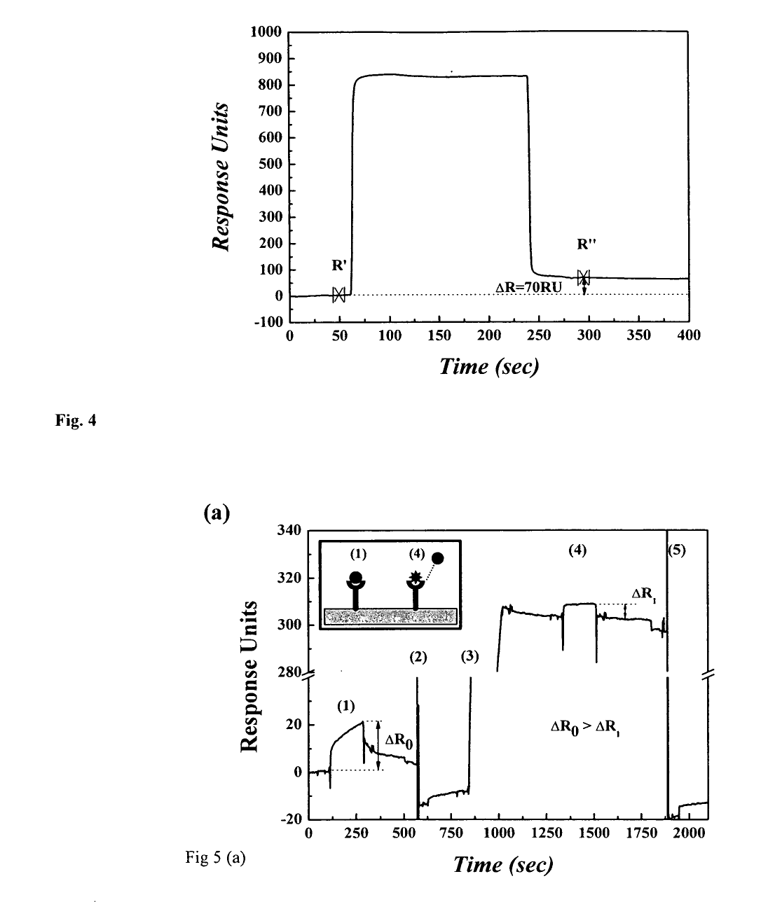

FIG. 4 shows a typical sensorgram for crude extract of Echinacea (CEE) binding CD28 immobilized on a chip (ΔR=˜851 RU). Most extract material is bound non-specifically and subsequently dissociates. The remaining extract material is bound specifically to CD28 (ΔR=˜70 RU) and could be removed by washing protocol (described previously). The sharp changes observed in ΔR across the time course indicate high affinity binding of CEE to the chip.

CEE can Block CD80 Binding CD28 Immobilised on the Chip

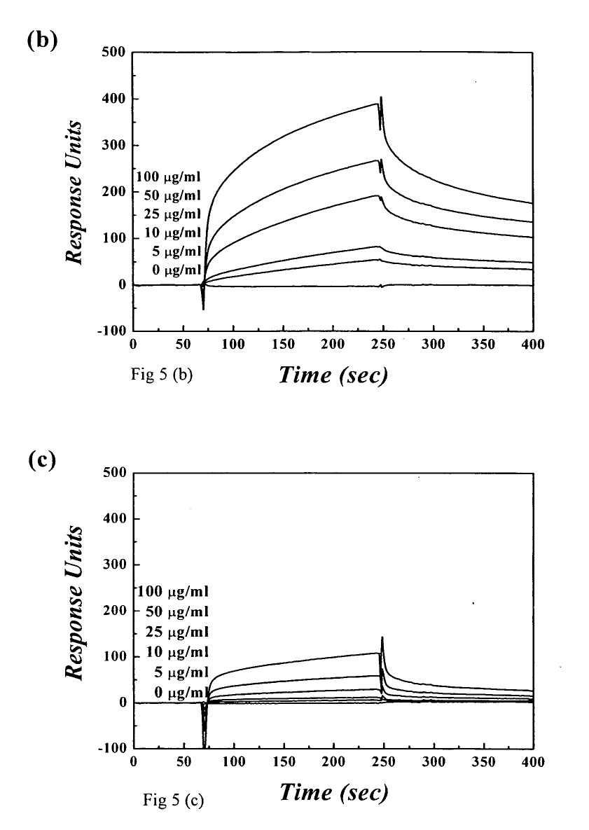

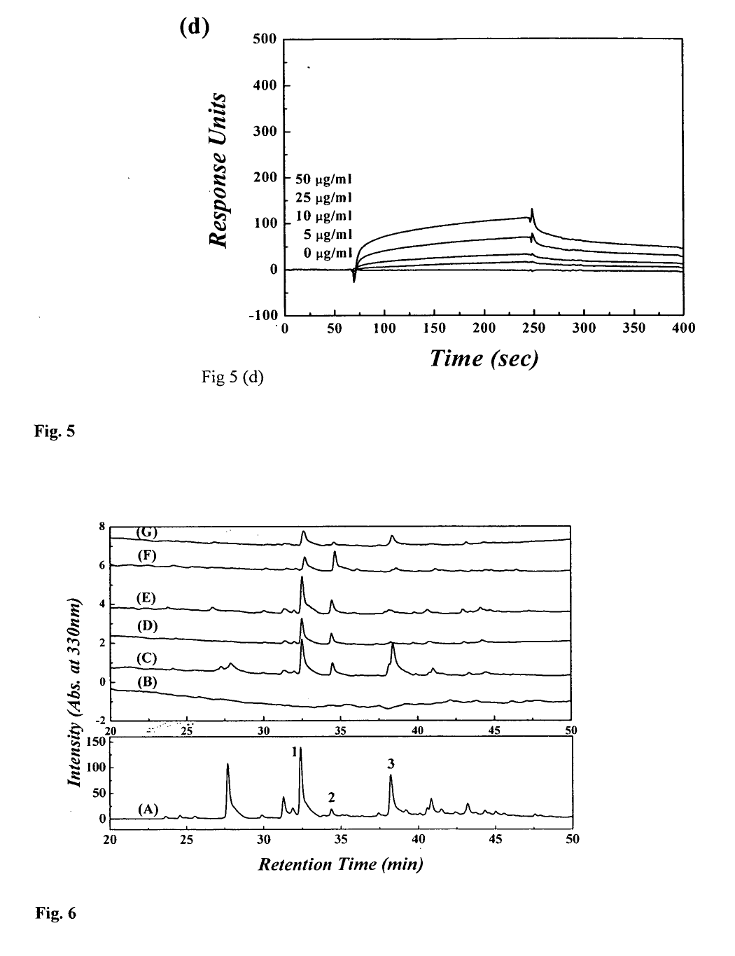

The following procedure was conducted: CD80 (10 μg/ml; 30 μl) was fluxed across and bound on the chip immobilized with CD28 (FIG. 5a; 1; ΔR0). Washing protocol was followed (FIG. 5a; 2). CEE was fluxed across, and bound on the chip (FIG. 5a; 3). CD80 (10 μg/ml; 30 μl) was again fluxed across the chip (FIG. 5a; 4; ΔR1). However, this time CD80 did not bind as well (ΔR0>ΔR1). The above procedure was repeated with anti-CD28 in place of CEE, with an equivalent result (ΔR0>ΔR1; data not shown). CEE and anti-CD28 evidently block CD80 binding (partially).

FIG. 5b shows the binding of differing concentrations of CD80 to immobilised CD28. FIG. 5c shows the binding of differing concentrations of CD80 to immobilised CD28, that has been previously fluxed with anti-CD28. FIG. 5d shows the binding of differing concentrations of CD80 to immobilised CD28, that has been previously fluxed with CEE. The RU as shown in FIGS. 5b-5d is the actual RU subtracted by the “background RU”. The “background RU” is the RU value without immobilised CD28 on the chip. Plots in FIGS. 5b-5d exhibit plateauing (saturation of binding) from which, with fitting (standard Langmuir binding isotherm), values of kₐ (association constant; M⁻¹) and k_{d} (dissociation constant; sec⁻¹) can be calculated. Affinity binding constants (1/K_{D}) can be obtained from K_{D,}, where K_{D} is calculated from Scatchard plots. Values are presented in Table 1.

TABLE 1
Kinetics values for CD80 binding chip immobilised CD28,
with or without prior anti-CD28 or crude extract of
Echinacea (CEE) binding to the CD28.

| | Parameters | | |
|---|---|---|---|
| Binding conditions | kₐ (M⁻¹) | k_{d} (sec⁻¹) | 1/K_{D} (M⁻¹) |
| imm-CD28->CD80 | 12.410³ | 10.410⁻³ | 9.110⁵ |
| imm-CD28->(anti-CD28)->CD80 | 2.910³ | 5.710⁻³ | 8.510⁴ |
| Imm-CD28->(CEE)->CD80 | 8.610³ | 4.210⁻³ | 4.910⁵ |

CD80 binds immobilised CD28 (1/K_{D}=9.1×10⁵ M⁻¹). This CD80-CD28 binding is weaker in the presence of anti-CD28 (1/K_{D}=8.5×10⁴ M⁻¹) or CEE (1/K_{D}=4.9×10⁵ M⁻¹). Quantitatively reiterating what was shown previously, CEE and anti-CD28 evidently block CD80 binding (partially).

HPLC Profiling of CEE

It has been shown here that a CEE component is able to selectively bind CD28, but what compound(s) constitutes this component? Bound CEE was washed off the chip and profiled with HPLC (FIG. 6(c-g); their similarity suggests reproducibility). There are three discernible peaks (demarked 1,2,3); three CEE compounds (C1,C2,C3) can bind CD28. Establishing that these bind the CD28 immobilised on the chip, and not the chip itself, CEE “wash off” from a chip with no immobilised receptor was profiled (FIG. 6(b)). In contrast, FIG. 6(a) shows the added complexity of spectrum obtained with CEE untreated by AFTIR (with CD28). For instance, in addition to the three peaks 1, 2 and 3, there are peaks 4 and 5. FIG. 7(a) shows this spectrum again, with FIGS. 7(b-e) showing the spectrums of isolated compounds C4, C1, C3 and C5 respectively. C2 could not easily be isolated. C1 and C3, being able to bind CD28, are hypothesised to have bioactivity, whilst C4 and C5 are not.

Bioassay of Compounds Highlighted by AFTIR

We have thus far established that components of CEE extract can selectively bind CD28. CD28 is a T cell transmembrane receptor, integral to this cell class's differentiation to the “active” state, a state that can be characterised by Interleukin-2 (IL-2) secretion. Can these CEE components bind CD28 in Jurkat cells, a T cell culture line, to downregulate IL-2 secretion i.e. can these CEE components act immuno-suppressively In vitro?

Jurkat cells can differentiate to the “active state” in response to one of two activatory signalling profiles: 1) Phorbol12-myristate 13-acetate (PMA) & lonomycin (CD-28 independent pathway); 2) anti-CD3 & anti-CD28 (CD-28 dependent pathway) (FIG. 8). These “unphysiological” signals stimulate intracellular signalling cascades that are believed to be physiologically relevant. The associated cascades: PMA→PKC→NF→kB; lonomycin→Ca²⁺→calmodulin→NF-AT; anti-CD28→CD28→Juk→Jun. Anti-CD3 works through three pathways: (i) anti-CD3→CD3/→TCR→MAPK→TF, (ii) anti-CD3→CD3/TCR→PLC→PKC→NF→kB, (iii) anti-D3→CD3/→TCR→Ca²⁺→calmodulin→NF-AT

Target transcription factors (NF-AT, NF-kB, TF, Jun) upregulate IL-2 expression.

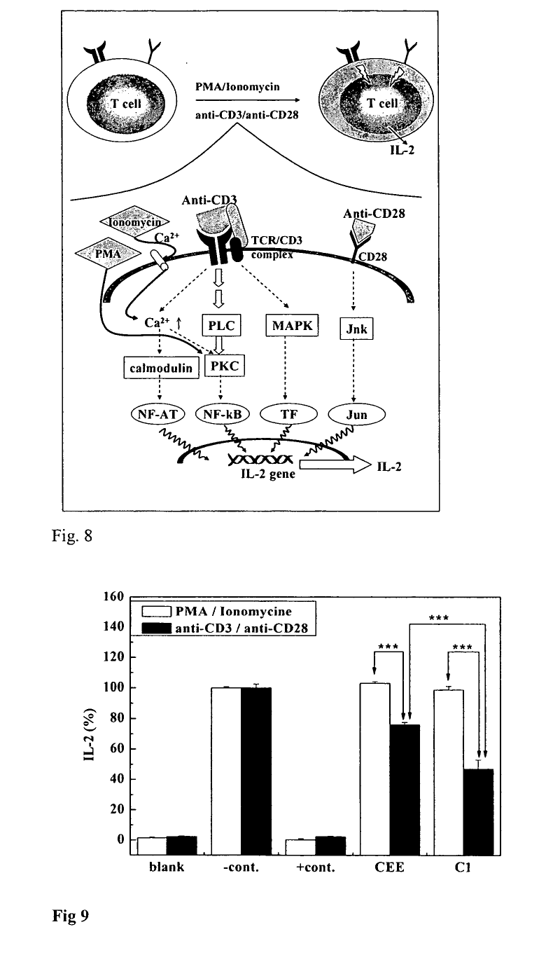

Jurkat cells (5x10⁵ cells/ml), treated with PBS buffer, produce little IL-2 (FIG. 9, blank). IL-2 secretion significantly increases with application of (PMA & lonomycin) or (anti-CD3 & anti-CD28) (FIG. 9. −cont. 1200 pg/μl of IL-2 released is normalized to 100%). This significant secretion with either of the activatory profiles is eradicated by the additional application of FK506, a clinical immunosuppressant (FIG. 9. +cont). CEE application does not modulate the IL-2 secretion response of Jurkat cells to PMA & lonomycin but does significantly downregulate (−22%) such response to anti-CD3 & anti-CD28 (FIG. 9. CEE). CEE can evidently disrupt CD28 dependent signaling in this In vitro preparation. None of compounds C2-C5 can modulate IL-2 response to either of the activatory profiles (data not shown). C1 however, although unable to modulate response to PMA & lonomycin, significantly downregulates (−53%) response to anti-CD3 & anti-CD28 (FIG. 9. C1). C1 downregulates IL-2 response more than CEE, likely due to its greater disruption of CD28 dependent signaling.

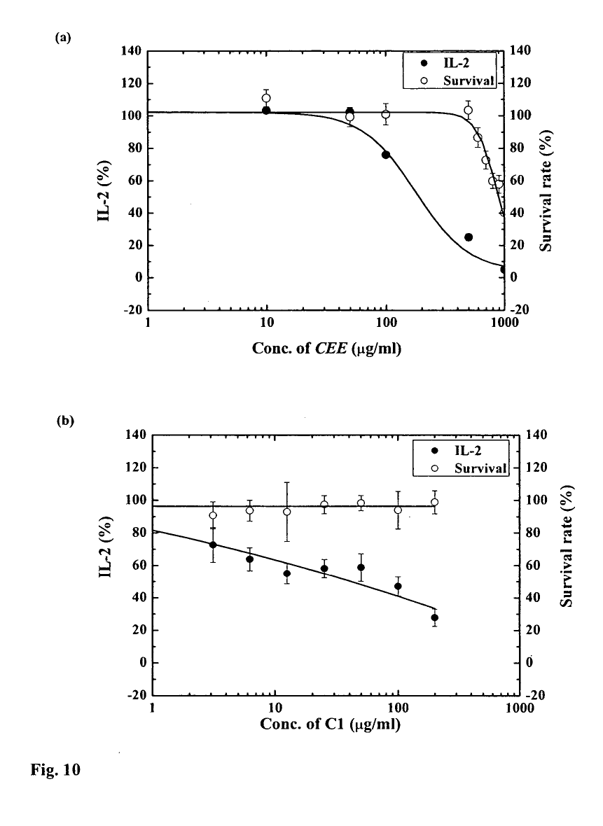

FIG. 10(a) shows 1) Jurkat IL-2 response to anti-CD3 & anti-CD28 (filled circles) and 2) Jurkat cell survival (%, open circles), in relation to the concentration of CEE applied. FIG. 10(b) shows the same dependent variables, but in relation to C1 concentration. CEE only inhibits IL-2 response at concentrations >10 μg/ml and is not toxic at concentrations <500 μg/ml. C1 linearly reduces IL-2 response, with no toxicity, across the concentration range investigated.

Chemical Identification of C1

C1 was chemically identified by mass spectrometry and NMR spectroscopy (¹³C and ¹H) as chlorogenic acid (5-O-caffeoylquinic acid) (see FIG. 11 for its chemical structure). C1 exhibits an [M+1]⁺ peak at m/z=355, indicating its molecular weight as that of chlorogenic acid. Chlorogenic acid has a quinic acid moiety. ¹³C NMR showed such a moiety in C1, with two methylenes (δ36.7, δ41.3), three oxymethines (δ68.3, δ73.0, δ74.8), one quaternary carbon (75.4), one carboxyl group (178.3). ¹H NMR signal from protons at C2, C5, C6, C7 and C8, in the 6-8 ppm region, closely approximated that of the caffeoyl moiety in chlorogenic acid (very slight shift). ¹³C NMR data also indicated the existence on the related chemical shift values.

Discussion

AFTIR (After Flowing Through Immobilized Receptor) is a novel method of screening herbal extracts for potential pharmaceuticals. As a proof of principle it was deployed to screen Echinacea for potential constituent immunosuppressants. CD28, a receptor instrumental to immune functioning, was covalently bound to a chip. Crucially, this binding was shown to be strong and non-impairing to native CD28 binding activity. Chip bound CD28 could still bind its antibody or CD80. Furthermore, these binding species could be dissociated by NaOH to yield the chip reusable, given that chip bound CD28 was shown to have maintained binding affinity after repeated binding—dissociation cycles.

Using this chip, an Echinacea component was shown to selectively bind CD28, partially blocking subsequent anti-CD28 and CD80 binding. HPLC profiling showed that this component consists of three compounds. One of which, identified by mass spectrometry and NMR spectroscopy as chlorogenic acid, was shown to be an immunosuppressant in vitro, downregulating CD28 dependent IL-2 expression in Jurkat cells (at non-toxic concentrations). Its hypothesised mechanism of action is its binding to extracellular CD28, disrupting CD28-CD80 binding and thus disturbing CD28 dependent T-cell activation. In vivo studies are needed to clarify further the potential of chlorogenic acid as a clinical immunosuppressant. It could perhaps prove medicinal in autoimmune or organ transplant cases. Echinacea purpurea was also shown to act immunosuppressantly in vitro, constituent chlorogenic acid being the likely conferment. Given Echinacea's wide pattern of use based on the pretext that it upregulates immune functioning, we found in contrast its adverse effect of immuno-suppressive action via this invention. Thus, such use of Echinacea might be moderately harmful. In clinical support, Echinacea prescription has been weakly associated with adverse effects in pediatric patients.

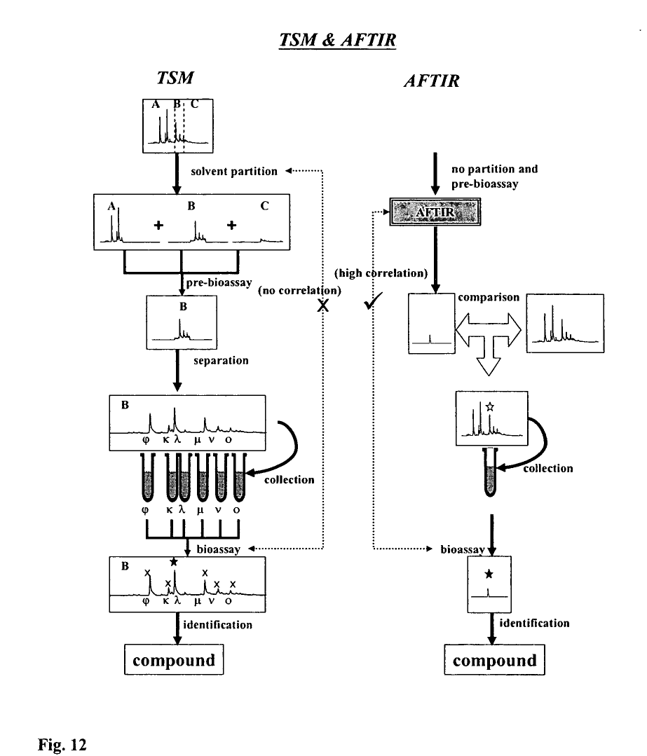

AFTIR is compared to the traditional screening method (TSM) in FIG. 12. Both use HPLC methodology to separate constituent extract compounds, such that they may be independently tested in a bioassay(s), for example the Jurkat IL-2 expression protocol in this report. However, AFTIR is more efficient as its partitioning step actually incorporates a bioassay, assessing whether any extract component can bind a rationally chosen receptor species. With AFTIR, only extract component that can bind receptor is profiled with HPLC, yielding a spectrum much reduced in complexity, greatly simplifying the HPLC process. In addition, AFTIR does not hinge on the unreliable assumption, integral to the TSM, that active ingredients are major peaks in the mass spectrum. AFTIR can detect trace components, minor peaks in the mass spectrum, with bioactivity.

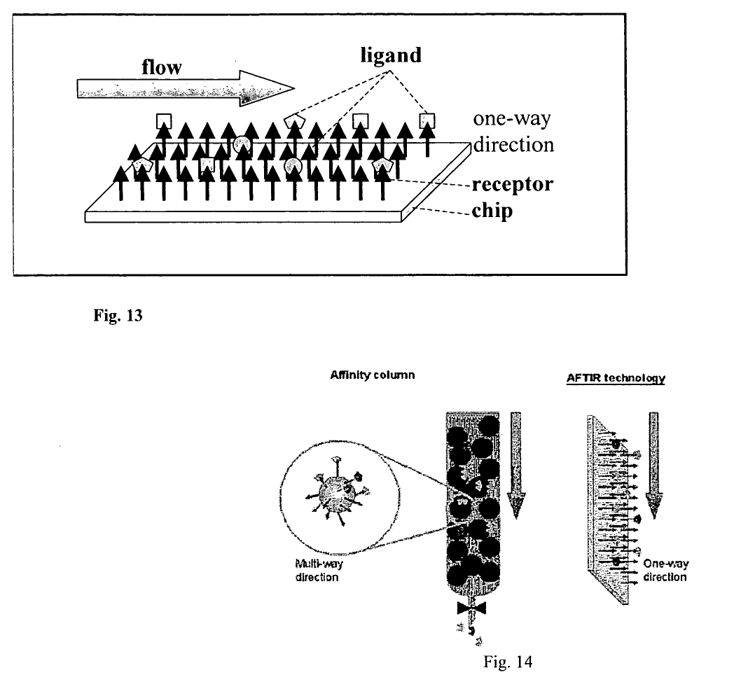

The full potential of AFTIR lies upon its principle being scaled (FIG. 13). Herbal extract being flown across an array of different chip-immobilised receptor species, bound components subsequently analysed; iterating for different herbal extracts. This approach is reductionist and perhaps misses some of the complexity in the action of herbal remedy. For example, it cannot capture the synergistic interactions of multiple herbal constituents. However, as reported here, it has proven promise nonetheless. In this report, AFTIR “fishes” with immune receptor “bait”, “catching” a candidate clinical immunosuppressant. Future studies could perhaps deploy AFTIR with “bait” receptors integral to cancer or inflammation, seeking anti-cancer and anti-inflammatory agents.

Compared to the Affinity Column (AC) method as discussed above, the present invention (AFTIR) here focuses more on the control of chip immobilized-receptor (flowing rate as a monitor for strong or weak binding control), screening efficiency, extending of sample sources such as a complex herbal composition. The differences between these two methods (AC and AFTIR) are further discussed as following:

(1) Setup of AFTIR

Setup of AC method includes the static phase for immobilized receptor and mobile phase for flowing sample (see FIG. 14, left side). In general, the static phase of AC containing the particle-like filling surface needs firstly stick with some particular materials such as Avidin, GST or antigen, etc. prior to the receptor having biotin, SH2 or its antibody can be immobilized. Upon flowing with sample mixture, the extent of binding will base on the different constituents of the sample in the affinity column. The separation effect is due to the length of the column. Both receptor-binding ability and separation effect of column are therefore equally contributed.

AFTIR also have both parts of static and mobile phases (see FIG. 14, right side). However, the static phase is constructed by using glass-base biochip with micro-flowing pathways rather than a huge column containing particles inside. The receptors (or fused with recombinant protein before immobilization) can be immobilized directly on the layer of carboxylated dextran in CM5 sensor chip by amine coupling with N-terminal (NH₂) of receptors. Upon flowing through the sample mixture, the materials with lower affinity pass over the chip and the targeted compounds bind to the receptor efficiently. A suitable buffer is used to dissociate the targeted compound(s) from the receptor. Subsequently, the collected targeted compound(s) can be run through HPLC again for the comparison with that of the raw sample. The targeting peak from AFTIR-HPLC spectra thereafter can be identified for the later analysis. All procedures can automatically be processed.

(2) Efficiency of AFTIR

In AFTIR in accordance with the present invention, the immobilized receptor can be fixed on chip with homology of orientation (or called it as one-way direction) (see FIG. 14, right side). This homological orientation of receptor in chip enhances the binding efficiency, which leads to the accuracy of finding the blocking materials. While, in AC method, the receptors will be immobilized in spherical type of particles (see FIG. 14, left side). The binding orientation of receptors in column is not much homological but with multi-way directions. Therefore, the efficiency of immobilized receptors to bind with the target ligands in AC is lower. For AFTIR, our results indicate that the binding constant (Kd) of the immobilized CD28 (imm-CD28) with ligand, CD80, is, about 1×10⁻⁸ M⁻¹ (see FIG. 5(b)), which is almost identical to that obtained from the literature. The ratio of binding between receptor (imm-CD28) and ligand (CD80) is about 1:0.92 (mole: mole). This implies that the one-way direction binding of AFTIR leads a high binding efficiency (>90%) with almost one to one binding type.

(3) Sensitivity of AFTIR

The amount of sample needed for both AC and AFTIR is different. In AC method, since the column is longer and its volume is bigger, the amount of receptors needed to fill the requirement is higher. This is particularly negative for the technique development by using these receptors isolated from cell membranes such as immune and cancer receptors because they are rare and very expensive. The small size of the chip used for AFTIR technique development indicates that the small amount of receptor is needed (in μg range) for running the experiment. FIG. 3 (b) shows the binding of anti-CD28 to imm-CD28. The sample used is only 10 μg/ml and the response (RU) shown in the figure is obvious. Therefore, a microarray chip with low doses but high applicability can be developed in accordance with the present invention.

(4) Selectivity of AFTIR

In AC method, the formula of target compound(s) is identified by mass spectrum, which is connected immediately after the affinity column. The test sample, which is required for AC, is needed to be simpler in order to identify target compound(s) based on the known database. Therefore, AC method is only suitable for simpler mixtures. For such a complex herbal medicine, AC method is not practical. While, the present invention, AFTIR, is intentionally designed for screening the target compound(s) from herbal medicines. The autochip model can enable the complex herbs be effectively screened and the desired compound(s) be obtained easily. Searching the useful/target compounds from wide spectrum of herbal medicines can therefore be accomplished in accordance with the present invention.

### EXAMPLE 2

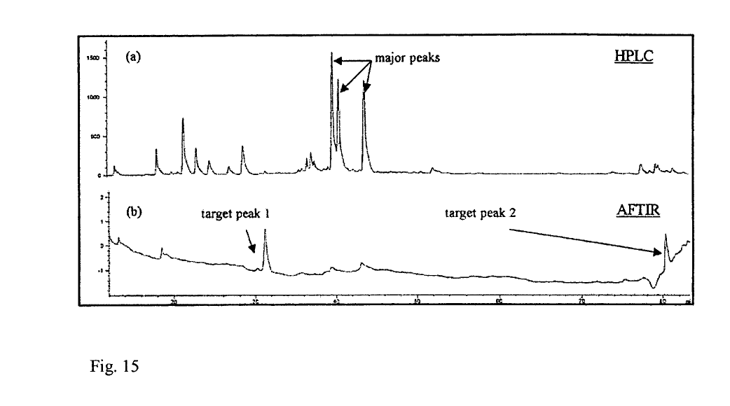

Similarly following the procedure as described in Example 1, a “crude” HPLC spectrum before “flowing though” and AFTIR spectrum of the “target” collection after “flowing though” in connection with another herbal extract, bidens pilosa, a popular Taiwan herb, were obtained (see FIG. 15).

Bidens pilosa was extracted by a solvent, n-butanol. The significant components of extract can be obtained from “crude” HPLC spectrum (see FIG. 15(a)): the extract (10 mg/ml; injection volume: 20 μl) was run though the RP-18 column by running buffer (0.05% TFA in H₂O and 0.05 TFA in CH₃N) with flowing rate of 0.6 ml/min and the detection wavelength at 254 nm. The CD 154 (a T-cell membrane receptor) was immobilized on the chip and the n-butanol extract was flowed though the chip. The “target” collection after “flowing though” was run through the column with the same conditions above for HPLC measurement. The results of AFTIR spectrum (the target collection measured by HPLC spectrum) is shown in FIG. 15b.

The HPLC spectrum (FIG. 15a) shows many absorption peaks with the major peak located at time domain around 40 min. While, the spectrum (FIG. 15b) obtained by AFTIR method seems to be simplified. The target peaks, indicating the ligands which are specifically bounded to the CD154, are shown in time domains of 32 min (target peak 1) and 81 min (target peak 2). The rest of signals are not outstanding. As compared with FIG. 15b, this implies that the main components of bidens pilosa (based on FIG. 15a) are not the desired targets for the identification of the compounds with immune function via CD154. On the contrary, the minor components shown at the time locations of target peaks 1 and 2 are desired to be identified. The AFTIR method therefore shows its usefulness and efficiency.

The invention is not limited by the embodiments described above which are presented as examples only but can be modified in various ways within the scope of protection defined by the appended patent claims.

We claim:

1. A method of identifying a target component of an herbal composition comprising:
immobilizing a plurality of receptors of interest on a chip;
flowing the herbal composition through the chip to allow binding of the target component with the receptors;
disassociating the bound target component from the immobilized receptors on the chip;
profiling the disassociated target component by HPLC; and
identifying the target component based on the HPLC profile of the disassociated target component.

2. The method of claim 1 wherein the chip is a glass-base biochip with micro-flowing pathways.

3. The method of claim 1 wherein the receptors are fused recombinant receptors.

4. The method of claim 1 wherein the receptors are immobilized on the chip with homology of orientation.

5. The method of claim 1 wherein the chip is a micro-array chip on which different types of receptor are immobilized.

6. The method of claim 1 wherein the chip is reused for binding the target component of the herbal composition after the bound target component is disassociated with the immobilized receptors on the chip.

7. The method of claim 1 wherein the disassociating step comprises washing the chip with a NaOH solution.

8. The method of claim 7 wherein the NaOH solution is 50 mM.

9. The method of claim 1 further comprising a step of identifying the target component by at least one of mass spectrometry and nuclear magnetic resonance, subsequent to the step of identifying by HPLC.

10. The method of claim 1 further comprising steps:
profiling the herbal composition by HPLC;
comparing the HPLC profile of the herbal composition with the HPLC profile of the disassociated target component to determine the peak corresponding to the target component in the HPLC profile of the herbal composition.

11. The method of claim 1 wherein the chip is selected from the group consisting of Sensor Chip CM5, CM4, CM3, C1, L1, SA, NTA, HPA, and Au.

12. The method of claim 1 wherein the chip is Sensor Chip CM5.

13. The method of claim 1 wherein the receptors are covalently immobilized on the chip.

14. The method of claim 13 wherein the chip is coated with a dextran having a carboxyl group, each of the receptors contains a N-terminal amino group, the receptors are immobilized on the chip through the bond of the carboxyl group and the N-terminal amino group.

15. The method of claim 1 wherein the molar ratio of binding between the target component and the receptor is at least 0.9:1.

16. The method of claim 1 wherein the minimum sufficient amount of the receptors is about 1 μg.

17. The method of claim 1 wherein the minimum sufficient amount of the herbal composition is about 1.5 mg.

18. The method of claim 1 wherein the receptors are the receptors that are involved in at least one of immunity, inflammation, and cancer.
